(19) United States
(12) Patent Application Publication
(10) Pub. No.: US 2024/0124818 A1
(43) Pub. Date: Apr. 18, 2024

(54) CELL CULTURE CONTAINER AND CELL CULTURE METHOD

(71) Applicant: TOYODA GOSEI CO., LTD., Kiyosu-shi, Aichi-ken (JP)

(72) Inventors: Seitaro TAKI, Kiyosu-shi, Aichi-ken (JP); Masateru YAMAZAKI, Kiyosu-shi, Aichi-ken (JP); Sayaka HINO, Nagoya-shi, Aichi-ken (JP)

(21) Appl. No.: 18/547,168

(22) PCT Filed: Feb. 22, 2022
(86) PCT No.: PCT/JP2022/007072

§ 371 Date: Aug. 21, 2023

(30) Foreign Application Priority Data
Mar. 25, 2021 (JP) 2021-050919

(51) Int. Cl. C12M 1/12; C12M 1/00; C12M 3/00

(52) U.S. Cl. CPC C12M 25/02; C12M 23/34; C12M 23/44; C12M 23/46

(57) ABSTRACT

A cell culture container includes a culture membrane held between first and second containers each including: a first surface which includes a holding portion for holding the culture membrane; a second surface which is opposite the first surface; a first cell and a second cell which are adjacent to each other in a second direction orthogonal to a first direction from the first surface toward the second surface; a partition wall which separates the first cell and the second cell and extends from the first surface toward the second surface; a communication portion which is formed between the partition wall and the second surface and causes the first cell and the second cell to communicate with each other; and a third surface which is opposite the partition wall in the second direction, defines the second cell together with the second surface and has an insertion opening formed.

### BACKGROUND

### Field

The present disclosure relates to cell culture containers and cell culture methods.

### Related Art

Conventionally, a cell culture apparatus is known that includes a culture membrane in which cells are cultured on both surfaces (for example, Patent Literature 1). In the cell culture apparatus of Patent Literature 1, the culture membrane is arranged at one end portion of an inner cylinder, and the other end portion of the inner cylinder is open. For example, when a tool such as a pipette is inserted into the inner cylinder, the tool is inserted from the other end portion toward the culture membrane.
[Patent Literature 1] Japanese Unexamined Patent Application Publication No. 2015-208283

### SUMMARY

### Technical Problem

However, in the cell culture apparatus disclosed in Patent Literature 1, the tool is inserted into the inner cylinder toward the culture membrane, and thus a tip end of the tool is highly likely to make contact with a cell membrane. When the tip end of the tool makes contact with the cell membrane, the cell membrane may be damaged. Hence, in a conventional technique, a careful operation is required so that the tip end of the tool does not make contact with the cell membrane, with the result that workability may be lowered.

### Solution to Problem

The present disclosure is able to be realized as aspects below.
(1) According to one aspect of the present disclosure, a cell culture container is provided. The cell culture container includes: a first container; a second container; and a culture membrane held between the first container and the second container, and each of the first container and the second container includes: a first surface which includes a holding portion for holding the culture membrane; a second surface which is opposite the first surface; a first cell and a second cell which are adjacent to each other in a second direction orthogonal to a first direction from the first surface toward the second surface; a partition wall which separates the first cell and the second cell and extends from the first surface toward the second surface; a communication portion which is formed between the partition wall and the second surface and causes the first cell and the second cell to communicate with each other; and a third surface which is opposite the partition wall in the second direction, defines the second cell together with the second surface and has an insertion opening formed. In the aspect described above, when a tool such as a pipette is inserted through the insertion opening, the possibility of contact of the pipette with the culture membrane is reduced by the partition. In this way, the extent to which the pipette is carefully operated so that the pipette does not make contact with the culture membrane is able to be reduced, and thus it is possible to enhance workability.
(2) In the cell culture container of the aspect described above, when the cell culture container is projected in the second direction, at least a part of the insertion opening may be formed in a position overlapping the communication portion. In the aspect described above, the tool is able to be inserted from the insertion opening through the communication portion along the second direction, with the result that it is possible to enhance workability.
(3) In the cell culture container of the aspect described above, the partition wall may further include a first cell surface which defines the first cell and is inclined such that as the first cell surface extends closer to the second surface, the first cell surface extends closer to the third surface. In the aspect described above, when the cell culture container is arranged in a first posture in which the first cell is located lower than the second cell, air mixed with a liquid put into the cell culture container is moved upward by buoyancy along the inclined first cell surface. Hence, the leaving of the air below the culture membrane is suppressed, and thus it Is possible to satisfactorily perform the culture on the culture membrane.
(4) In the cell culture container of the aspect described above, the first cell may further include a second cell surface which is opposite the partition wall, extends from the first surface toward the second surface and is inclined such that as the second cell surface extends closer to the second surface, the second cell surface extends away from the third surface. In the aspect described above, when the cell culture container is arranged in the first posture in which the first cell is located lower than the second cell, the liquid put into the cell culture container is moved downward by gravity along the inclined second cell surface. The liquid put into the cell culture container is stored on a lower portion of the second cell surface of the first cell, and thus it is possible to easily suck out the liquid.
(5) In the cell culture container of the aspect described above, at least one of the first container and the second container may further include a fourth surface which is opposite the second cell surface and supports the second cell surface such that in a posture of the cell culture container in which the fourth surface is placed on a horizontal plane, the second cell surface is inclined with respect to a horizontal direction. In the aspect described above, the fourth surface of the cell culture container is placed on the horizontal plane, and thus the second cell surface is inclined with respect to the horizontal plane, with the result that it is possible to enhance the workability of an operation of sucking out the liquid or the like.
(6) A cell culture method using the cell culture container of the aspect described above may include: a first step of arranging the cell culture container in a first posture in which the first cell is located lower than the second cell and injecting a first cell suspension into the first container via the insertion opening of the first container; a second step of arranging, after the first step, the cell culture container in a second posture in which the first container is located higher than the second container and keeping the cell culture container in the second posture until a first cell included in the first cell suspension is fixed to the culture membrane; a third step of arranging, after the second step, the cell culture container in the first posture and keeping the cell culture container in the first posture to continue culture of the first cell; a fourth step of injecting, after the third step, a second cell suspension into the second container of the cell culture container arranged in the first posture via the insertion opening of the second container; a fifth step of arranging, after the fourth step, the cell culture container in a third posture in which the second container is located higher than the first container and keeping the cell culture container in the third posture until a second cell included in the second cell suspension is fixed to the culture membrane; and a sixth step of arranging, after the fifth step, the cell culture container in the first posture and keeping the cell culture container in the first posture to continue culture of the second cell. In the aspect described above, it is possible to satisfactorily co-culture cells in the cell culture container of the aspect described above.

### BRIEF DESCRIPTION OF THE DRAWINGS

FIG. 1 is an exploded perspective view of a cell culture container;

FIG. 2 is a side view of the cell culture container in partial cross section;

FIG. 3 is a flowchart showing steps in a liquid-air cell culture method;

FIG. 4 is a schematic view of the cell culture container corresponding to a first step;

FIG. 5 is a schematic view of the cell culture container corresponding to a second step;

FIG. 6 is a schematic view of the cell culture container corresponding to a third step;

FIG. 7 is a schematic view of the cell culture container corresponding to a fourth step;

FIG. 8 is a diagram showing the cell culture container which is being turned to a third posture;

FIG. 9 is a schematic view of the cell culture container corresponding to a fifth step;

FIG. 10 is a schematic view of the cell culture container corresponding to a sixth step; and

FIG. 11 is a flowchart showing steps in a liquid-liquid cell culture method.

### DETAILED DESCRIPTION

### A. Embodiment

A1. Configuration of Cell Culture Container.

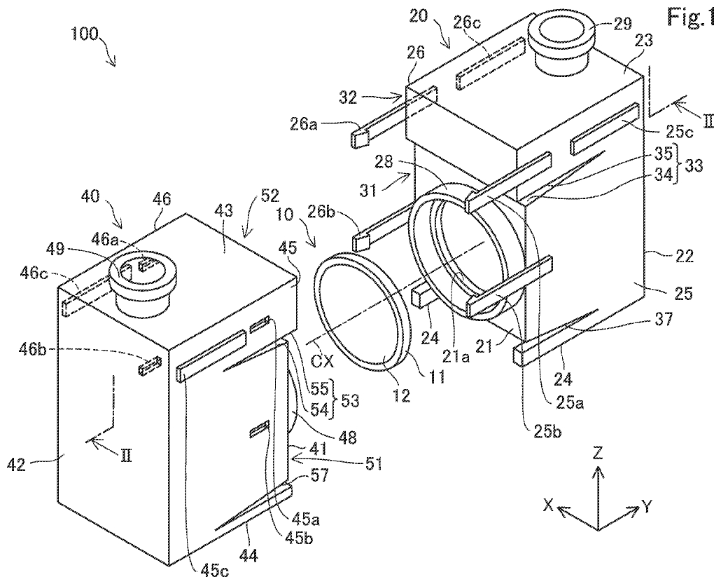

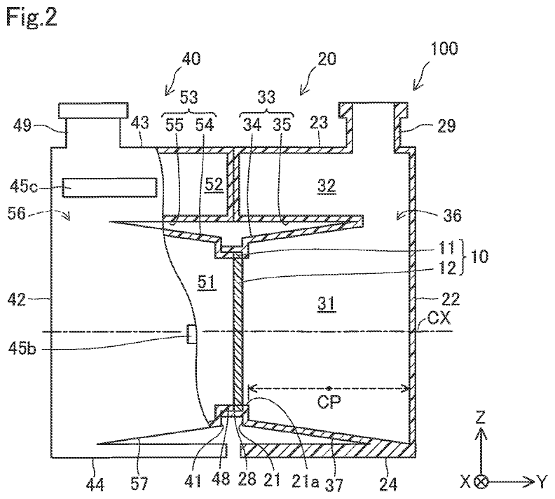

FIG. 1 is an exploded perspective view of a cell culture container 100. FIG. 2 is a side view of the cell culture container 100 in partial cross section. The cross section shown in FIG. 2 is a cross section taken along line II-II in FIG. 1. In FIGS. 1 and 2, an X axis, a Y axis and a Z axis which are orthogonal to each other are shown. The cell culture container 100 is typically arranged such that a Z direction is along a vertical direction, and is used. In the following description, a +Z direction may be assumed to be an upward direction, and a −Z direction may be assumed to be a lower direction. The cell culture container 100 includes a culture membrane 10, a first container 20 and a second container 40. In the cell culture container 100 used for cell culture, in a state where the culture membrane 10 is sandwiched between the first container 20 and the second container 40, they are integrated to be used. The cell culture container 100 is a container which is used for co-culturing cells on both surfaces of the culture membrane 10. The first container 20 and the second container 40 are substantially plane-symmetrical. The first container 20 and the second container 40 are formed of, for example, a hard resin material. Examples of the hard resin material include polystyrene, polycarbonate, an acrylic resin and the like, and a transparent resin is particularly preferable.

As shown in FIG. 1, the culture membrane 10 includes a membrane support portion 11 and a cell culture membrane 12. The membrane support portion 11 is an annular frame. The circular cell culture membrane 12 is joined to the membrane support portion 11. The cell culture membrane 12 is a membrane which serves as a scaffold for cell culture when cell culture is performed. As the cell culture membrane 12, a porous membrane made of a synthetic polymer or a porous membrane made of a natural polymer which has a large number of pores penetrating in the thickness direction of the membrane may be used. Specific examples of the porous membrane include a track etching membrane, a nanofiber membrane, a collagen vitrigel membrane, a porous polyurethane membrane and the like. As the porous polyurethane membrane, for example, a porous polyurethane membrane is mentioned which is manufactured using a manufacturing method described substantially and including a first step and a second step. The first step is a step of forming, on a substrate, a layer of an uncured polyurethane raw material. The second step is a step of supplying water vapor to an exposed surface of the polyurethane raw material layer formed on the substrate which is separated from the substrate so as to facilitate the curing of the polyurethane raw material in the polyurethane raw material layer and to form a porous shape having a plurality of projections and recesses in the exposed surface.

The first container 20 is in a substantially rectangular parallelepiped external shape, and has an internal space. The first container 20 includes a first surface 21, a second surface 22, a third surface 23, a fourth surface 24, a fifth surface 25, a sixth surface 26, a holding portion 28 and an insertion opening 29. When the first container 20 is integrated with the second container 40, the first surface 21 is opposite the second container 40, and includes the holding portion 28 for holding the culture membrane 10. The holding portion 28 is annular, and is provided to protrude in a −Y direction orthogonal to the first surface 21 with the first surface 21 serving as a base end. In the first surface 21, a hole 21a having a smaller diameter than the inner circumferential surface of the holding portion 28 is formed along the inner circumferential surface of the holding portion 28. Hence, the internal space of the first container 20 communicates with the outside via the holding portion 28. As shown in FIG. 2, when the first container 20 is integrated with the second container 40, the culture membrane 10 is arranged inside the holding portion 28 and outside the first surface 21. The diameter of the hole 21a is smaller than the diameter of the inner circumferential surface of the holding portion 28. Hence, the first surface 21 protrudes in a radial direction with respect to the inner circumferential surface of the holding portion 28 toward the center axis CX of the holding portion 28. In this way, the movement of the culture membrane 10 to the side of the internal space of the first container 20 is restricted.

As shown in FIG. 1, the second surface 22 is opposite the first surface 21 in a Y direction. The direction from the first surface 21 toward the second surface 22 is also referred to as a first direction. In the first container 20 of the present embodiment, a +Y direction is the first direction. The third surface 23 and the fourth surface 24 intersect the first surface 21 and the second surface 22. Here, “two surfaces intersect each other” means a state where the two surfaces intersect each other or either of a state where an extension of one surface intersects the other surface and a state where extensions of the two surfaces intersect each other. The third surface 23 and the fourth surface 24 are opposite each other in the Z direction. The fifth surface 25 and the sixth surface 26 intersect the first surface 21, the second surface 22, the third surface 23 and the fourth surface 24. The fifth surface 25 and the sixth surface 26 are opposite each other.

In the third surface 23, the insertion opening 29 is provided. The insertion opening 29 is used when a tool such as a pipette is inserted into the first container 20. The insertion opening 29 is in a cylindrical shape, and is provided to protrude in the +Z direction orthogonal to the third surface 23 with the third surface 23 serving as a base end. In the third surface 23, a hole is formed along the inner circumferential surface of the insertion opening 29. Hence, the internal space of the first container 20 communicates with the outside via the insertion opening 29. The thickness of the side wall of the insertion opening 29 is not uniform, and the outer circumferential surface of a tip end portion protrudes outward in a radial direction with respect to the outer circumferential surface of a base end portion. In this way, it is possible to suppress falling off of a first lid 201, which will be described later, when the first lid 201 is attached.

In the fifth surface 25, a first locking portion 25a, a second locking portion 25b and a lid locking portion 25c are formed. The first locking portion 25a and the second locking portion 25b have the same shape. The first locking portion 25a is formed on an upper side with respect to the center of the fifth surface 25 in the Z direction. The first locking portion 25a is formed on the fifth surface 25 in a position closer to the first surface 21 than to the second surface 22 in the Y direction. The first locking portion 25a is in the shape of a long plate in the Y direction, and a base end portion is fixed to a fifth surface 45. A tip end of the first locking portion 25a protrudes from the edge of the fifth surface 45 in the −Y direction. At the tip end of the first locking portion 25a, a hook is formed which engages with the first locking recess portion 45a of the second container 40. Specifically, the tip end of the first locking portion 25a protrudes in a +X direction. The second locking portion 25b is arranged lower than the first locking portion 25a. The lid locking portion 25c is a projection portion for locking a second lid 202 which will be described later. The position of the lid locking portion 25c in the Z direction is substantially the same as that of the first locking portion 25a. The lid locking portion 25c is formed on the fifth surface 25 in a position closer to the second surface 22 than to the first surface 21 in the Y direction. The lid locking portion 25c is in a long rectangular shape in the Y direction, and is provided on the fifth surface 25 to protrude. As on the fifth surface 25, on a sixth surface 46, a first locking portion 26a, a second locking portion 26b and a lid locking portion 26c are formed. The first locking portion 26a, the second locking portion 26b and the lid locking portion 26c are respectively formed in positions plane-symmetric with those of the first locking portion 25a, the second locking portion 25b and the lid locking portion 25c with respect to a YZ plane including the center axis CX. Since the first locking portion 26a, the second locking portion 26b and the lid locking portion 26c respectively have the same shapes as the first locking portion 25a, the second locking portion 25b and the lid locking portion 25c, the description thereof is omitted.

As shown in FIG. 2, the first container 20 includes, in addition to the configuration described above, a first cell 31, a second cell 32, a partition wall 33 and a communication portion 36. The first cell 31 and the second cell 32 are spaces which are formed by separating the internal space of the first container 20 with the partition wall 33. The first cell 31 and the second cell 32 are adjacent to each other in the Z direction which is a second direction orthogonal to the +Y direction which is the first direction from the first surface 21 toward the second surface 22. The second cell 32 is separated with the third surface 23 opposite the partition wall 33 in the second direction and the second surface 22. The partition wall 33 extends in a direction from the first surface 21 toward the second surface 22 with the first surface 21 serving as a base end. A tip end of the partition wall 33 is separated from the second surface 22. In the present embodiment, the tip end of the partition wall 33 is located between a center portion CP between the inner surface of the first surface 21 and the inner surface of the second surface 22 and the second surface 22.

The partition wall 33 includes a first cell surface 34 and a third cell surface 35. The first cell 31 further includes a second cell surface 37. The second cell surface 37 is opposite the partition wall 33. The first cell surface 34 and the second cell surface 37 separate the first cell 31. The first cell surface 34 is inclined such that as the first cell surface 34 extends closer to the second surface 22, the first cell surface 34 extends closer to the third surface 23. In this way, as described in detail later, when a liquid is put into the first cell 31, air mixed with the liquid is able to easily escape.

The communication portion 36 is formed between the partition wall 33 and the second surface 22, and causes the first cell 31 and the second cell 32 to communicate with each other. In other words, the communication portion 36 is said to be a gap between the tip end of the partition wall 33 and the second surface 22. The first cell 31 and the second cell 32 communicate with each other only via the communication portion 36. In the present embodiment, the communication portion 36 is located between the center portion CP and the inner surface of the second surface 22 in the first direction, that is, in the +Y direction with respect to the first container 20.

The second cell surface 37 couples the first surface 21 and the fourth surface 24 together. The second cell surface 37 is inclined such that as the second cell surface 37 extends closer to the second surface 22, the second cell surface 37 extends away from the third surface 23. In other words, the second cell surface 37 is inclined such that in a first posture in which the fourth surface 24 is placed on a horizontal plane to serve as a bottom surface and to cause the third surface 23 to serve as an upper surface, as the second cell surface 37 extends closer to the second surface 22, the second cell surface 37 extends lower. In this way, as described in detail later, it is possible to easily suck out the liquid stored in the first cell 31. In the first posture, the first cell 31 is located lower than the second cell 32. In the first posture, as described later, a pipette 300 is inserted from the insertion opening 29 to inject a first cell suspension 401.

In the present embodiment, a gap is formed between the second cell surface 37 and the fourth surface 24. As shown in FIG. 1, in the fourth surface 24, a center portion in an X direction is cut out, and thus an end portion of the first container 20 in the +X direction and an end portion in a −X direction are formed. The fourth surface 24 is formed, and thus in the first posture of the cell culture container 100, a state where the first cell surface 34 and the second cell surface 37 are inclined with respect to the horizontal plane is able to be easily kept.

As shown in FIG. 2, when the cell culture container 100 is projected in the Z direction which is the second direction orthogonal to the first direction from the first surface 21 toward the second surface 22, at least a part of the insertion opening 29 is formed in a position overlapping the communication portion 36. In this way, a tool such as the pipette 300 is able to be inserted from the insertion opening 29 through the communication portion 36 substantially along the second direction, with the result that it is possible to enhance workability. In the present embodiment, when the cell culture container 100 is projected, in the first direction, that is, the +Y direction with respect to the first container 20, the insertion opening 29 is located between the center portion CP between the inner surface of the first surface 21 and the inner surface of the second surface 22 and the second surface 22.

As shown in FIG. 1, as with the first container 20, the second container 40 includes a first surface 41, a second surface 42, a third surface 43, a fourth surface 44, a fifth surface 45, a sixth surface 46, a holding portion 48 and an insertion opening 49. The holding portion 48 of the second container 40 differs in size from the holding portion 28 of the first container 20. Specifically, the outside diameter of the annular holding portion 48 is smaller than the inside diameter of the holding portion 28. As shown in FIG. 2, when the first container 20 and the second container 40 are integrated, the holding portion 48 is covered by the holding portion 28. The outside diameter of the holding portion 48 is substantially the same as that of the culture membrane 10. In a state where the culture membrane 10 is sandwiched between the end surface of the holding portion 48 in the +Y direction and the first surface 21 inside the holding portion 28 of the first container 20, the first container 20 and the second container 40 are integrated. In this way, the culture membrane 10 is held by the first container 20 and the second container 40.

As shown in FIG. 1, in the fifth surface 45, the first locking recess portion 45a, a second locking recess portion 45b and a lid locking portion 45c are formed. Since the lid locking portion 45c has the same configuration as the lid locking portion 25c related to the first container 20, the description thereof is omitted. The first locking recess portion 45a and the second locking recess portion 45b are respectively formed in positions which correspond to the tip ends of the first locking portion 25a and the second locking portion 25b when the first container 20 and the second container 40 are integrated. The first locking recess portion 45a and the second locking recess portion 45b each are in a long rectangular shape in the Y direction and are recessed with respect to the fifth surface 25. When the first container 20 and the second container 40 are integrated, the tip end of the first locking portion 25a is fitted to the first locking recess portion 45a, and the tip end of the second locking portion 25b is fitted to the second locking recess portion 45b. In the sixth surface 46, as in the fifth surface 25, a first locking recess portion 46a and a second locking recess portion 46b corresponding to the first locking portion 26a and the second locking portion 26b of the first container 20 are formed. When the first container 20 and the second container 40 are integrated, the tip ends of the first locking portion 26a and the second locking portion 26b are respectively fitted to the first locking recess portion 46a and the second locking recess portion 46b. As described above, the first locking portions 25a and 26a and the second locking portions 25b and 26b of the first container 20 respectively engage with the first locking recess portions 45a and 46a and the second locking recess portions 45b and 46b of the second container 40, and thus the first container 20 and the second container 40 are integrated. For example, when the culture membrane 10 is replaced, the engagement of the first container 20 and the second container 40 is released, and when the culture is performed, the first container 20 and the second container 40 engage with each other, and thus it is possible to handle the first container 20 and the second container 40 together. Hence, it is possible to enhance the workability of an operation using the cell culture container 100.

Since the first surface 41, the second surface 42, the third surface 43, the fourth surface 44, the fifth surface 45, the sixth surface 46 and the insertion opening 49 respectively have the same configurations as the first surface 21, the second surface 22, the third surface 23, the fourth surface 24, the fifth surface 25, the sixth surface 26 and the insertion opening 29 of the first container 20, the description thereof is omitted. As shown in FIG. 2, the second container 40 includes, in addition to the configuration described above, a first cell 51, a second cell 52, a partition wall 53 and a communication portion 56. The partition wall 53 includes a first cell surface 54 and a third cell surface 55. The first cell 51 further includes a second cell surface 57. Since the first cell 51, the second cell 52, the partition wall 53, the communication portion 56 and the second cell surface 57 respectively have the same configurations as the first cell 31, the second cell 32, the partition wall 33, the communication portion 36 and the second cell surface 37 of the first container 20, the description thereof is omitted.

A2. Liquid-Air Cell Culture Method Using Cell Culture Container:

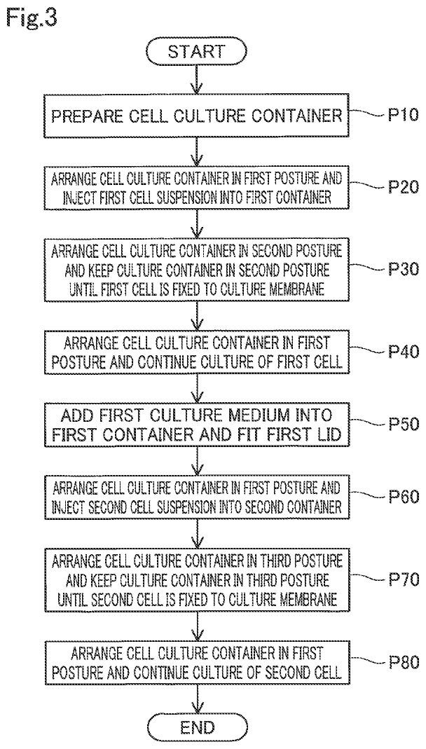

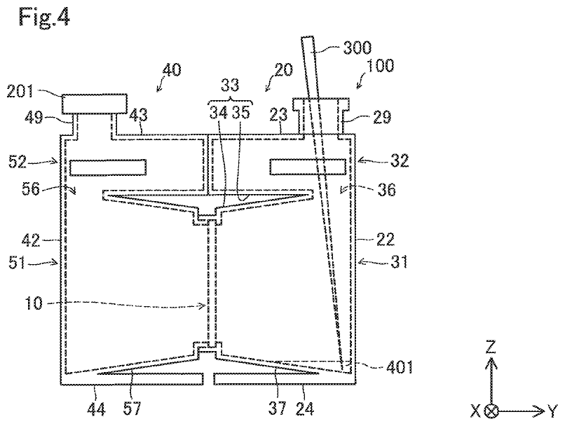

A liquid-air cell culture method using the cell culture container 100 will be described. FIG. 3 is a flowchart showing steps in the liquid-air cell culture method of cells using the cell culture container 100. FIGS. 4 to 10 are schematic views of the cell culture container 100 for description of the steps shown in FIG. 3. In FIGS. 4 to 10, an X axis, a Y axis and a Z axis in the use of the cell culture container 100 are shown. A Z direction indicates a vertical direction, and an XY plane indicates a horizontal plane. A +Z direction and a −Z direction are also referred to as an upward direction and a downward direction, respectively.

In the step P10 of FIG. 3, the cell culture container 100 is first prepared which is shown in FIG. 4 and in which the first lid 201 is fitted to the insertion opening 49 of the second container 40. The first lid 201 is made of, for example, an elastic material such as a soft resin or an elastomer. Examples of the soft resin include LDPE (Low Density Polyethylene) and PP (polypropylene). Examples of the elastomer include silicone rubber, polyurethane, olefinic thermoplastic elastomers and the like. The first lid 201 is in a shape to cover the insertion opening 49. When the first lid 201 is attached to the insertion opening 49, the first lid 201 is extended and fitted to cover the insertion opening 49. In this way, the end surface of the insertion opening 49 in the Z direction is in intimate contact with the first lid 201, and thus the second container 40 is kept airtight.

In the first step P20 of FIG. 3, the cell culture container 100 is arranged in a first posture (injection posture) in which the first cell 31 is located lower than the second cell 32. In the first posture, the first cell suspension 401 is injected into the first container 20 via the insertion opening 29 of the first container 20. The first cell suspension 401 includes a first cell 402 and a first culture medium which is a liquid for the first cell 402. The first cell suspension 401 is injected, and thus the first cell 402 is seeded on the culture membrane 10. As shown in FIG. 4, in the first step P20, the pipette 300 is used to inject the first cell suspension 401. Here, the pipette 300 is inserted from the insertion opening 29 through the communication portion 36 such that the tip end reaches the vicinity of the fourth surface 24. The culture membrane 10 is arranged on the first surface 21 which intersects the fourth surface, and the movement of the pipette 300 toward the culture membrane 10 is restricted by the partition wall 33. Hence, it is possible to suppress the contact of the pipette 300 and the culture membrane 10. In this way, it is possible to suppress damage to the culture membrane 10. Although in the present embodiment, an operator performs the operation using the pipette 300, the injection operation may be automatically performed without human intervention using a device or the like. In the cell culture container 100 of the present embodiment, the movement of a tool toward the culture membrane 10 is restricted by the partition wall 33. Hence, high positional accuracy in the operation of the tool for preventing the culture membrane 10 from being damaged is not required, and it is possible to automatically perform the operation. In this way, it is possible to reduce an operation time. After the injection of the first cell suspension 401, the first lid 201 is fitted to the insertion opening 29.

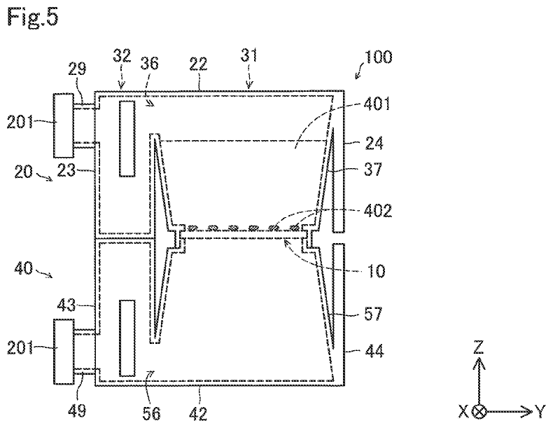

In the second step P30 of FIG. 3, as shown in FIG. 5, the cell culture container 100 is arranged in a second posture (first fixing posture) in which the first container 20 is located higher than the second container 40, and the cell culture container 100 is kept in the second posture until the first cell 402 included in the first cell suspension 401 is fixed to the culture membrane 10. In the second posture, the second surface 42 of the second container 40 serves as a bottom surface, and the second surface 22 of the first container 20 serves as an upper surface.

In the second step P30, the cell culture container 100 is turned such that the second posture shown in FIG. 5 is achieved from the state of the first posture shown in FIG. 4. The first lid 201 is fitted to the insertion opening 29, and thus outflow of the first cell suspension 401 from the insertion opening 29 is suppressed. Although FIG. 5 shows a state where the first cell suspension 401 is stored only in the first cell 31, for example, when a large amount of first cell suspension 401 is used, the first cell suspension 401 may also be stored in the second cell 32. The membrane surface of the culture membrane 10 is arranged in a horizontal direction, and thus the fixing of the first cell 402 to the culture membrane 10 is facilitated. A time period until the first cell 402 is fixed to the culture membrane 10 is, for example, about half a day to one day.

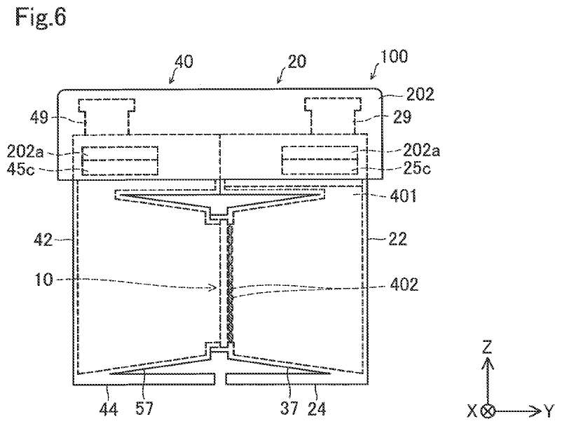

In the third step P40 of FIG. 3, the cell culture container 100 is arranged in the first posture, the cell culture container 100 is kept in the first posture and thus the culture of the first cell 402 is continued. In the third step P40, the cell culture container 100 is turned such that the first posture shown in FIG. 6 is achieved from the state of the second posture shown in FIG. 5. Thereafter, the first lids 201 fitted to the insertion openings 29 and 49 are removed, and the second lid 202 is fitted.

Here, the second lid 202 will be described. The second lid 202 is made of a hard resin material. Examples of the hard resin material include polystyrene, polycarbonate, an acrylic resin and the like, and a transparent resin is particularly preferable. The second lid 202 is fitted to the cell culture container 100 to cover the insertion openings 29 and 49. The second lid 202 is in the shape of a box open toward the −Z direction. In the inner peripheral surface of the second lid 202, four protrusion portions 202a are formed which protrude inward. The four protrusion portions 202a are respectively formed in positions which correspond to the lid locking portions 25c, 26c, 45c and 46c of the cell culture container 100. In this way, the four protrusion portions 202a respectively abut against the lid locking portions 25c, 26c, 45c and 46c, and thus the second lid 202 is arranged in a state where the bottom surface of the second lid 202 is separated from the insertion openings 29 and 49. The second lid 202 is fitted, and thus contamination during the culture is suppressed. Since the second lid 202 is fitted to the cell culture container 100 in a state where the second lid 202 is separated from the end surfaces of the insertion openings 29 and 49, a state where the internal space of the cell culture container 100 communicates with the outside is kept. In this way, it is possible to replace a gas in the internal space of the cell culture container 100. During the culture, the cell culture container 100 is placed in an incubator adjusted to a target oxygen concentration or carbon dioxide concentration. The first culture medium is replaced as necessary, and the first cells 402 are cultured until they are confluent. The culture period is, for example, about several days.

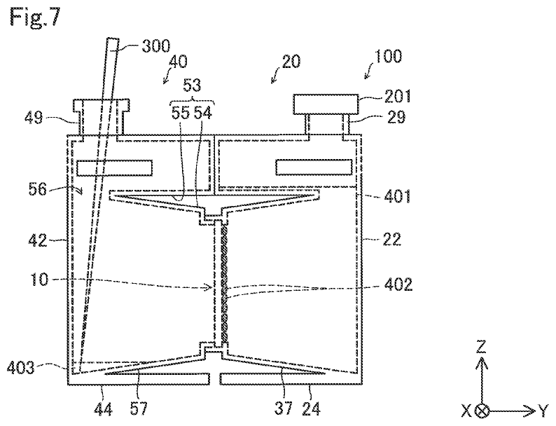

In the step P50 of FIG. 3, the second lid 202 is removed, the first culture medium is added into the first cell 31 and the first lid 201 is fitted to the insertion opening 29. In a fourth step P60, a second cell suspension 403 is injected into the second container 40 of the cell culture container 100 arranged in the first posture via the insertion opening 49 of the second container 40. The second cell suspension 403 includes a second cell 404 and a second culture medium which is a liquid for the second cell 404. The second cell suspension 403 is injected, and thus the second cell 404 included in the second cell suspension 403 is seeded on the culture membrane 10. As shown in FIG. 7, in the fourth step P60, as in the first step P20, the pipette 300 is used to inject the second cell suspension 403. The culture membrane 10 is arranged on the first surface 41 which intersects the fourth surface 44, and the movement of the pipette 300 toward the culture membrane 10 is restricted by the partition wall 53. Hence, it is possible to suppress the contact of the pipette 300 and the culture membrane 10. After the injection of the second cell suspension 403, the first lid 201 is fitted to the insertion opening 49.

In the fifth step P70 of FIG. 3, the cell culture container 100 is arranged in a third posture (second fixing posture) in which the second container 40 is located higher than the first container 20, and the cell culture container 100 is kept in the third posture until the second cell 404 included in the second cell suspension 403 is fixed to the culture membrane 10. In the third posture, the second surface 22 of the first container 20 serves as a bottom surface, and the second surface 42 of the second container 40 serves as an upper surface.

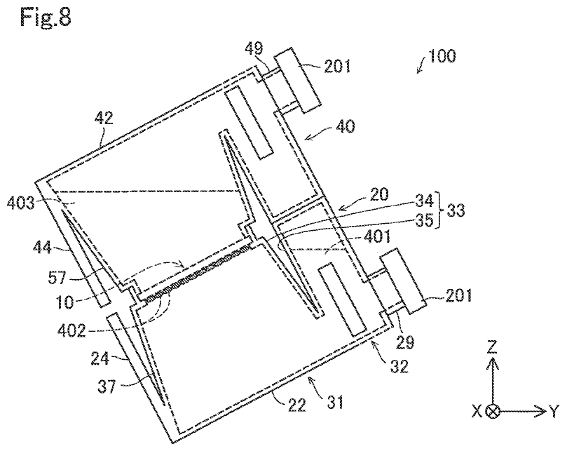

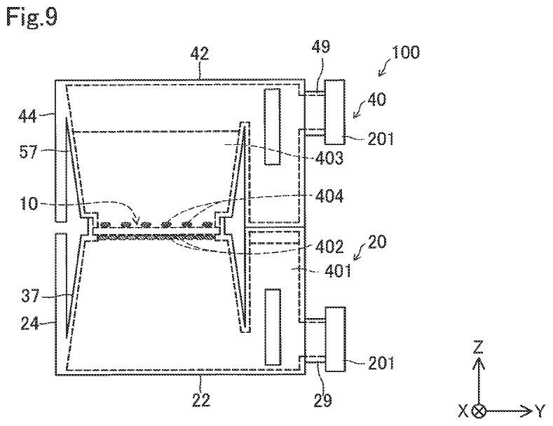

FIG. 8 is a diagram showing a state where the cell culture container 100 is being turned from the first posture to the third posture. In the first container 20, the first cell suspension 401 is stored in a state where a space is left in an upper portion. The air in the space moves upward in the first container 20 as the cell culture container 100 is turned. Here, when the internal space of the first container 20 is not separated by the partition wall 33, the air moves below the culture membrane 10. In this respect, in the present embodiment, the internal space of the first container 20 is separated by the partition wall 33, and thus the air is stored in the second cell 32. In this way, the leaving of the air below the culture membrane 10 is suppressed, and thus the first cell 402 is kept in contact with the first culture medium. Furthermore, when the cell culture container 100 is turned, even if bubbles are generated in the first cell suspension 401, the bubbles are stored in the second cell 32. Hence, it is possible to satisfactorily perform the culture. In the third posture shown in FIG. 9, the membrane surface of the culture membrane 10 is arranged in the horizontal direction, and thus the fixing of the second cell 404 to the culture membrane 10 is facilitated. A time period until the second cell 404 is fixed to the culture membrane 10 is, for example, about half a day to one day.

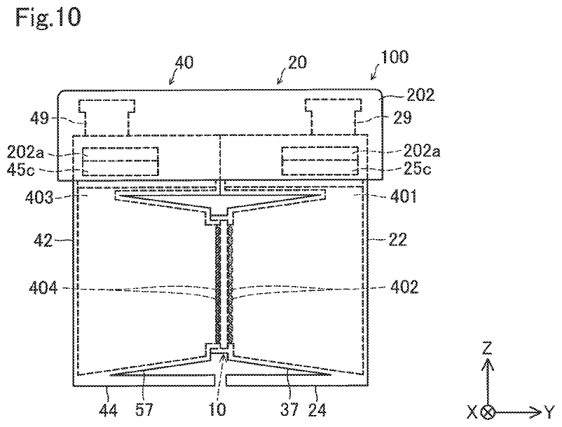

In the sixth step P80 of FIG. 3, the cell culture container 100 is arranged in the first posture, the cell culture container 100 is kept in the first posture and thus the culture of the second cell 404 is continued. In the sixth step P80, the cell culture container 100 is turned such that the first posture shown in FIG. 10 is achieved from the state of the third posture shown in FIG. 9. Thereafter, the first lids 201 fitted to the insertion openings 29 and 49 are removed, and the second lid 202 is fitted. As in the third step P40, the second culture medium is replaced as necessary, and the second cells 404 are cultured until they are confluent. The culture period is, for example, about several days. When the culture of the second cell 404 is completed, the present culture step is completed.

When after the completion of the culture, transepithelial electrical resistance (TEER) is measured to evaluate the barrier properties of the culture membrane 10 in which the first cell 402 and the second cell 404 are cultured, two TEER electrodes are inserted through the insertion openings 29 and 49. In the cell culture container 100, the insertion openings 29 and 49 are formed opposite each other through the culture membrane 10. Hence, the two TEER electrodes are inserted through the insertion openings 29 and 49, and thus it is possible to easily perform the measurement. The measurement of the transepithelial electrical resistance is not limited to the measurement after the completion of the culture step. For example, the transepithelial electrical resistance may be measure after the first cell 402 is cultured on the culture membrane 10 in a state where the first culture medium is injected into the second container 40. In other words, it is possible to measure the TEER even in a state where the cells are cultured only on one surface of the culture membrane 10.

A3. Liquid-Liquid Cell Culture Method Using Cell Culture Container:

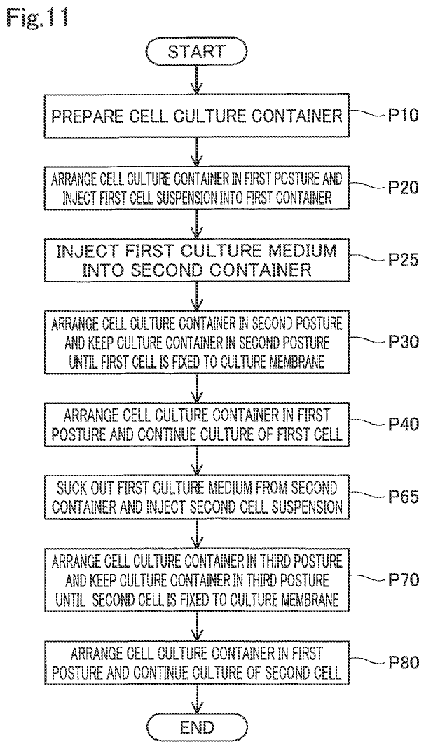

A liquid-liquid cell culture method using the cell culture container 100 will be described. FIG. 11 is a flowchart showing steps in the liquid-liquid cell culture method of cells using the cell culture container 100. The same steps as in FIG. 3 are identified with the same symbols, and the detailed description thereof is omitted as necessary.

After the first cell suspension 401 is injected into the first container 20 in the first step P20, in a step P25, the first culture medium is injected into the second container 40 with the pipette 300. In this way, in a state where both surfaces of the cell culture membrane 12 are in contact with the first culture medium, the culture of the first cell 402 is able to be performed. Thereafter, the second step P30 and the third step P40 are performed, and the first cells 402 are cultured until they are confluent. In a step P65, in order to culture the second cell 404, the first culture medium is sucked out from the second container 40, for example, with the pipette 300, and the second cell suspension 403 is injected. The step P65 is also referred to as the fourth step. Thereafter, the first lid 201 is fitted to the insertion opening 49. Here, the second cell surface 57 is inclined, and thus the first culture medium is stored on a lower portion of the second cell surface 57. Thus, it is possible to easily suck out the first culture medium from the second container 40. The suction of the first culture medium may be performed with an aspirator. In the cell culture container 100 according to the present embodiment, the movement of the tool toward the culture membrane 10 is restricted by the partition wall 33. Hence, high positional accuracy in the operation of the tool for preventing the culture membrane 10 from being damaged is not required, and it is possible to automatically perform the operation. Thereafter, the fifth step P70 and the sixth step P80 are performed, and thus the second cells are cultured until they are confluent, with the result that the present culture step is completed.

In the embodiment described above, the first container 20 includes the partition wall 33 which separates the first cell 31 and the second cell 32, and the second container 40 includes the partition wall 53 which defines the first cell 51 and the second cell 52. In this way, when the pipette 300 or the like is inserted through the insertion opening 29 or 49, the possibility of contact of the pipette 300 with the culture membrane 10 is reduced by the partition 33 or 53. Hence, the extent to which the pipette 300 is carefully operated so that the pipette 300 does not make contact with the culture membrane 10 is able to be reduced, and thus it is possible to enhance workability. When the cell culture container 100 is projected in the second direction orthogonal to the first direction from the first surface 21 toward the second surface 22, at least a part of the insertion opening 29 is formed in a position overlapping the communication portion 36. In this way, the pipette 300 is able to be inserted from the insertion opening 29 through the communication portion 36 along the second direction, with the result that it is possible to enhance workability. The partition wall 33 includes the first cell surface 34 which is inclined such that as the first cell surface 34 extends closer to the second surface 22, the first cell surface 34 extends closer to the third surface 23. In this way, when the cell culture container 100 is arranged in the first posture, the leaving of the air below the culture membrane 10 is suppressed, and thus it is possible to satisfactorily perform the culture on the culture membrane 10. The first cell 31 includes the second cell surface 37 which is inclined such that as the second cell surface 37 extends closer to the second surface 22, the second cell surface 37 extends away from the third surface 23. In this way, the liquid put into the cell culture container 100 is stored on a lower portion of the second cell surface 37 of the first cell 31, with the result that it is possible to easily suck out the liquid. The cell culture container 100 includes the fourth surface 24 which supports the second cell surface 37 such that in a posture of the cell culture container 100 in which the fourth surface 24 is placed on a horizontal plane, the second cell surface 37 is inclined with respect to the horizontal direction. In this way, the fourth surface 24 of the cell culture container 100 is placed on the horizontal plane, and thus the second cell surface 37 is inclined with respect to the horizontal plane, with the result that it is possible to enhance the workability of an operation of sucking out the liquid or the like. With the cell culture method described above including the steps from the first step P20 to the sixth step P80 and using the cell culture container 100, it is possible to satisfactorily co-culture cells in the cell culture container configured as described above.

### B. Other Embodiments

(B1) In the embodiment described above, in both the first container 20 and the second container 40, the fourth surfaces 24 and 44 are formed. By contrast, in one of the first container 20 and the second container 40, one of the fourth surfaces 24 and 44 may be formed. Since the first container 20 and the second container 40 are integrated to be used, one of the first container 20 and the second container 40 includes the fourth surface 24, and thus the second cell surface 37 or 57 is able to be kept inclined with respect to the horizontal plane.

(B2) In the embodiment described above, the second cell surface 37 is inclined to the second surface 22. By contrast, the second cell surface 37 may be inclined partway to the second surface 22. Even when a part of the second cell surface 37 is inclined, the liquid is able to be collected on the part, and thus it is possible to easily suck out the liquid.

(B3) In the embodiment described above, the tip end of the partition wall 33 is located between the center portion CP and the second surface 22. By contrast, the tip end of the partition wall 33 may be located between the center portion CP and the first surface 21 or at the center portion CP in the first direction. Regardless of the position of the tip end of the partition wall 33, the partition wall 33 is provided, and thus when the tool is inserted from the insertion opening 29, the possibility of contact of the tool with the culture membrane 10 is able to be reduced. The same is true for the partition wall 53. However, in the embodiment described above, the following positional relationship is preferably provided on the positions of the insertion opening 29, the partition wall 33 and the culture membrane 10. Specifically, the partition wall 33 is preferably located on a line connecting the opening of the tip end of the insertion opening 29 and the culture membrane 10 (any part of the culture membrane 10). In this way, when a rod-shaped tool such as the pipette 300 is inserted into the cell culture container 100 from the insertion opening 29, the partition wall 33 is able to serve as a barrier to more reliably prevent the tool from making contact with the culture membrane 10.

The present disclosure is not limited to the embodiments described above, and may be realized in various configurations without departing from the spirit thereof. For example, the technical features of embodiments corresponding to the technical features of aspects described in the “SUMMARY” may be replaced or combined as necessary in order to solve a part or all of the problems described above or in order to achieve a part or all of the effects described above. When the technical features are not described as essential features in the present specification, they may be deleted as necessary.
10: culture membrane, 11: membrane support portion, 12: cell culture membrane, 20: first container, 21, 41: first surface, 21a: hole, 22, 42: second surface, 23, 43: third surface, 24, 44: fourth surface, 25, 45: fifth surface, 25a: first locking portion, 25b: second locking portion, 25c, 26c, 45c, 46c: lid locking portion, 26, 46: sixth surface, 26a: first locking portion, 26b: second locking portion, 28, 48: holding portion, 29, 49: insertion opening, 31, 51: first cell, 32, 52: second cell, 33, 53: partition wall, 34, 54: first cell surface, 35, 55: third cell surface, 36, 56: communication portion, 37, 57: second cell surface, 40: second container, 45a: first locking recess portion, 45b: second locking recess portion, 46a: first locking recess portion, 46b: second locking recess portion, 100: cell culture container, 201: first lid, 202: second lid, 202a: protrusion portion, 300: pipette, 401: first cell suspension, 402: first cell, 403: second cell suspension, 404: second cell, CP: center portion, CX: center axis, P10, P25, P50, P65: step, P20: first step, P30: second step, P40: third step, P60: fourth step, P70: fifth step, P80: sixth step

1. A cell culture container comprising:
a first container;
a second container; and
a culture membrane held between the first container and the second container,
wherein each of the first container and the second container includes:
a first surface which includes a holding portion for holding the culture membrane;
a second surface which is opposite the first surface;
a first cell and a second cell which are adjacent to each other in a second direction orthogonal to a first direction from the first surface toward the second surface;
a partition wall which separates the first cell and the second cell and extends from the first surface toward the second surface;
a communication portion which is formed between the partition wall and the second surface and causes the first cell and the second cell to communicate with each other; and
a third surface which is opposite the partition wall in the second direction, defines the second cell together with the second surface and has an insertion opening formed.

2. The cell culture container according to claim 1,
wherein when the cell culture container is projected in the second direction, at least a part of the insertion opening is formed in a position overlapping the communication portion.

3. The cell culture container according to claim 1,
wherein the partition wall further includes
a first cell surface which defines the first cell and is inclined such that as the first cell surface extends closer to the second surface, the first cell surface extends closer to the third surface.

4. The cell culture container according to claim 1,
wherein the first cell further includes
a second cell surface which is opposite the partition wall, extends from the first surface toward the second surface and is inclined such that as the second cell surface extends closer to the second surface, the second cell surface extends away from the third surface.

5. The cell culture container according to claim 4,
wherein at least one of the first container and the second container further includes
a fourth surface which is opposite the second cell surface and supports the second cell surface such that in a posture of the cell culture container in which the fourth surface is placed on a horizontal plane, the second cell surface is inclined with respect to a horizontal direction.

6. A cell culture method using the cell culture container according to claim 1, the cell culture method comprising:
a first step of arranging the cell culture container in a first posture in which the first cell is located lower than the second cell and injecting a first cell suspension into the first container via the insertion opening of the first container;
a second step of arranging, after the first step, the cell culture container in a second posture in which the first container is located higher than the second container and keeping the cell culture container in the second posture until a first cell included in the first cell suspension is fixed to the culture membrane;
a third step of arranging, after the second step, the cell culture container in the first posture and keeping the cell culture container in the first posture to continue culture of the first cell;
a fourth step of injecting, after the third step, a second cell suspension into the second container of the cell culture container arranged in the first posture via the insertion opening of the second container,
a fifth step of arranging, after the fourth step, the cell culture container in a third posture in which the second container is located higher than the first container and keeping the cell culture container in the third posture until a second cell included in the second cell suspension is fixed to the culture membrane; and
a sixth step of arranging, after the fifth step, the cell culture container in the first posture and keeping the cell culture container in the first posture to continue culture of the second cell.
